(19) United States
(12) Patent Application Publication
(10) Pub. No.: US 2007/0287160 A1
(43) Pub. Date: Dec. 13, 2007

(54) Methods for high throughput screening of cell lines

(76) Inventors: Judy H. Chou, Carlsbad, CA (US); Peter Franchi, Lynnfield, MA (US); Sarah Koob, Andover, MA (US); Nadine Barron, Tewksbury, MA (US)

Correspondence Address: WilmerHale/Wyeth, 60 STATE STREET, BOSTON, MA, 02109, US

(21) Appl. No.: 11/788,815

(22) Filed: Apr. 20, 2007

Related U.S. Application Data
(60) Provisional application No. 60/793,991, filed on Apr. 21, 2006.

(51) Int. Cl. G01N 33/53

(52) U.S. Cl. 435/7.2

(57) ABSTRACT

Disclosed are methods for high-throughput screening of cell lines for use in protein expression in certain pharmaceutical, drug development, and biotechnological processes such that high productivity cell lines are identified for their ability to produce both desired levels of protein expression and appropriate quality of a protein-of-interest.

This Application claims the benefit of priority to U.S. Provisional Application No. 60/793,991, filed Apr. 21, 2006, the specification of which is incorporated by reference in its entirety.

### FIELD OF THE INVENTION

The present invention relates generally to the field of pharmaceuticals. More specifically, the invention pertains to methods for screening cell lines for the ability to produce sufficient quantities and comparable quality of protein for industrial-scale production.

### BACKGROUND OF THE INVENTION

High-throughput technology has become an important tool in pharmaceutical and biotechnology research. High-throughput analytical methodologies utilize automated procedures to rapidly analyze the activity of proteins, the level of protein expression, gene expression, and the myriad of chemical interactions that occurs in a biological system. The data generated by these methodologies and technologies has been put to use in a wide range of fields such as cancer research, drug discovery, and crystallography (see, e.g., Abramovitz et al. (2006) Proteome Sci. 4(1): 5 [Epub ahead of print]).

High-throughput analyses depend on the ability to sense a particular chemical interaction or compound out of a vast array of chemical reactions occurring in a system. High-throughput technologies have been used to probe the specific chemical interactions and levels of expression of thousands of genes in a short period of time. To accomplish this difficult task, certain techniques have been developed that utilize molecular signals (e.g., fluorophores) and automated analyses that process information at a very rapid rate (see, e.g., Pinhasov et al. (2004) Comb. Chem. High Throughput Screen. 7(2):133-40). For example, microarray technology has been extensively utilized to probe the interactions of thousands of genes at once, while providing valuable information for specific genes (see, e.g., Mocellin and Rossi (2007) Adv. Exp. Med. Biol. 593:19-30).

In the past few years, both automated and manual high-throughput protein expression and purification has become an accessible means to rapidly screen and produce soluble proteins for structural and functional studies (see Cabrita et al. (2006) BMC Biotechnol. 6: 12). Developments such as filter-plate based assays for cloning, expression and purification, ligation-independent cloning (LIC), and auto-induction for protein expression have been joined with automated systems to create parallel production techniques of proteins that are simple and cost-effective (see Cabrita et al. (2006) BMC Biotechnol. 6: 12; Aslanidis et al. (1990) Nucleic Acids Res. 18(20): 6069-6074). These approaches have provided researchers with powerful tools to produce proteins at industrial-scale levels for use in pharmaceuticals and drug development.

However, the purification and expression of proteins from particular cells presents particular difficulties. Once a protein-of-interest has been identified, it is rather difficult to obtain significant quantities of that protein in a purified form. As a result, cell lines producing recombinant proteins are used to produce proteins in sufficient quantities for industrial development.

Although cell lines can provide researchers with the opportunity to produce large quantities of a particular protein, they are not always efficient producers of proteins. Certain cell line clones may produce sub-optimal levels of protein. Other cell line clones may produce optimal levels of protein expression, but fail to produce a fully functional pool of protein due to either structural malformation or inappropriate post-translational modification. Such issues can lead to wasted time and resources during development of a biologically relevant compound. Therefore, high-throughput screening methodologies that rapidly and reliably provide information on the quality and quantity of a protein expressed in a particular cell line are needed. The present invention is directed to these and other important ends.

### SUMMARY OF THE INVENTION

By analyzing the levels of expression of a protein-of-interest in a cell line prior to starting full scale production of the protein-of-interest, significant time and resources will not be wasted on cell lines that are of insufficient quality for high-throughput protein expression. The present invention is based, in part, upon the discovery that high-throughput screening procedures and high-throughput purification procedures can be performed to determine the cell lines that produce sufficient quantities of a protein-of-interest. In addition, these procedures can be utilized to screen the candidate cell lines to determine whether they produce the protein-of-interest with a desired quality, for example, biological activity. These high-throughput screening and high-throughput-purification techniques (hereinafter together referred to as “high-throughput screening”) are therefore valuable methods for determining the proper cell lines to utilize in industrial-scale protein expression. This discovery has been exploited to provide an invention that allows for the use of high-throughput-screening methods to identify cell lines that are useful for industrial-scale production of a protein-of-interest.

In one aspect, the invention provides a method of high-throughput screening of cell lines for protein expression. The method includes the high-throughput titer screening of cell lines to determine the level of protein expression in each cell line. Cell lines that produce a desired level of protein expression are selected for subsequent high-throughput purification of the protein. The cell line is selected for protein expression if the cell line produces a desired level of protein expression and it has an appropriate quality.

In some embodiments, the protein is an antibody, ligand, receptor, subunit of protein, fragment of protein, fusion protein, recombinant protein, and fragment of the same. In certain embodiments, the protein is an antibody, a recombinant antibody, or a F(ab′)2 fragment.

In other embodiments, the first binding agent is selected from the group consisting of antibodies, ligands, receptors, fusion proteins, subunits of proteins, recombinant proteins, and fragments of the same. In particular embodiments, the first binding agent is Protein A or streptavidin. In still other embodiments, the binding agents can be attached to a solid support such as beads, plates, and microarray chips. In many embodiments, the solid support comprises cellulose, sepharose, polyacrylamide, glass, or polystyrene.

In other embodiments, the second binding agent is selected from the group consisting of antibodies, ligands, receptors, fusion proteins, subunits of proteins, recombinant proteins, and fragments of the same. In particular embodiments, the second binding agent is an antibody and fragments of the same. In more particular embodiments, the antibody is a F(ab′)2 fragment, and is even more particularly an F(ab′)2 fragment that specifically binds to the Fc portion of an antibody.

In still other embodiments, the detectable label is a fluorophore, chemical dye, radioactive binding agent, chemiluminescent binding agent, electrochemiluminescent agent, magnetic binding agent, paramagnetic binding agent, promagnetic binding agent, enzyme that yield a colored product, enzyme that yield a chemiluminescent product, and enzyme that yield a magnetic product. In very particular embodiments, the detectable label is ruthenium or multiple ruthenium labels.

In some embodiments, the reagent comprises a resin, which, in particular embodiments, has a third binding agent attached to it. In certain embodiments, the third binding agent is selected from the group consisting of antibodies, ligands, receptors, fusion proteins, subunits of proteins, recombinant proteins, and fragments of the same. While in particular embodiments, the third binding agent is Protein A or streptavidin. In some embodiments, the protein is eluted from the reagent using a vacuum. In other embodiments, the protein is eluted by centrifugation. In still other embodiments, the protein is eluted by gravity flow through the resin. In many embodiments, the screening of the incubated cell lines utilizes an automated workstation.

In another aspect, the invention provides a method of high-throughput screening of cell lines for protein expression. The method includes a step of incubating cell lines in media, and contacting a solid support with a sample from each incubated cell line. In this aspect, the solid support has attached to its surface a first binding agent that binds to a protein in each sample. The protein that is bound by the first binding agent is contacted with a second binding agent, which is operably linked to a detectable label, that binds to the protein. The level of protein expression in each sample is determined by detecting the label operably linked to the second binding agent bound to the protein. The method also includes the selection of the cell lines that have a desired level of protein expression. In some instances, this will be determined by comparing the level of protein expression in each cell line to the average level of protein expression in all of the cell lines. For instance, an increased level of protein expression as compared to the average level of expression in all cell lines could be the desired level of protein expression. Alternatively, a decreased level of protein expression could be the desired level of protein expression, which would also be determined by comparing the level of protein expression in each screened cell line to the average level of protein expression in all cell lines.

The method of this aspect of the invention further entails isolating supernatants from the selected cell lines, and distributing each supernatant to a well of a multiwell plate. The supernatants are then contacted with a reagent that binds to the protein. The protein is eluted from the reagent and assayed for appropriate quality. According to this aspect of the invention, a cell line is selected for protein expression if the cell line was selected in step e) and the protein expressed by the cell line has appropriate quality. In some embodiments, the protein is an antibody, ligand, receptor, subunit of protein, fragment of protein, fusion protein, recombinant protein, and fragment of the same. In certain embodiments, the protein is an antibody, a recombinant antibody, or a F(ab′)2 fragment.

In other embodiments, the first binding agent is selected from the group consisting of antibodies, ligands, receptors, fusion proteins, subunits of proteins, recombinant proteins, and fragments of the same. In particular embodiments, the first binding agent is Protein A or streptavidin. In still other embodiments, the binding agents can be attached to a solid support such as beads, plates, and microarray chips. In many embodiments, the solid support comprises cellulose, sepharose, polyacrylamide, glass, or polystyrene.

In other embodiments, the second binding agent is selected from the group consisting of antibodies, ligands, receptors, fusion proteins, subunits of proteins, recombinant proteins, and fragments of the same. In particular embodiments, the second binding agent is an antibody and fragments of the same. In more particular embodiments, the antibody is a F(ab′)2 fragment, and is even more particularly an F(ab′)2 fragment that specifically binds to the Fc portion of an antibody.

In still other embodiments, the detectable label is a fluorophore, chemical dye, radioactive binding agent, chemiluminescent binding agent, electrochemiluminescent agent, magnetic binding agent, paramagnetic binding agent, promagnetic binding agent, enzyme that yield a colored product, enzyme that yield a chemiluminescent product, and enzyme that yield a magnetic product. In very particular embodiments, the detectable label is ruthenium or multiple ruthenium labels.

In some embodiments, the reagent comprises a resin, which, in particular embodiments, has a third binding agent attached to it. In certain embodiments, the third binding agent is selected from the group consisting of antibodies, ligands, receptors, fusion proteins, subunits of proteins, recombinant proteins, and fragments of the same. While in particular embodiments, the third binding agent is Protein A or streptavidin. In some embodiments, the protein is eluted from the reagent using a vacuum. In many embodiments, the screening of the incubated cell lines utilizes an automated workstation.

In another aspect, the invention provides a method of cell culture process development. The method includes a step of incubating each cell line in a different condition to be tested. Cell line samples are then placed into contact with a solid support from each cell line from each different condition. In this aspect, the solid support has attached to its surface a first binding agent that binds to a protein in each sample. The protein that is bound by the first binding agent is contacted with a second binding agent, which is operably linked to a detectable label, that binds to the protein. The level of protein expression in each sample is determined by detecting the label operably linked to the second binding agent bound to the protein. The method also includes the selection of the cell lines that have a desired level of protein expression. In some instances, this will be determined by comparing the level of protein expression in each cell line to the average level of protein expression in all of the cell lines. For instance, an increased level of protein expression as compared to the average level of expression in all cell lines could be the desired level of protein expression. Alternatively, a decreased level of protein expression could be the desired level of protein expression, which would also be determined by comparing the level of protein expression in each screened cell line to the average level of protein expression in all cell lines.

The method of this aspect of the invention further entails isolating supernatants from the selected cell lines, and distributing each supernatant to a well of a multiwell plate. The supernatants are then contacted with a reagent that binds to the protein. The protein is eluted from the reagent and assayed for appropriate quality. According to this aspect of the invention, a proper cell culture condition is identified if the protein quantity and quality are improved as compared to other conditions tested.

In other embodiments, the first binding agent is selected from the group consisting of antibodies, ligands, receptors, fusion proteins, subunits of proteins, recombinant proteins, and fragments of the same. In particular embodiments, the first binding agent is Protein A or streptavidin. In still other embodiments, the binding agents can be attached to a solid support such as beads, plates, and microarray chips. In many embodiments, the solid support comprises cellulose, sepharose, polyacrylamide, glass, or polystyrene.

In other embodiments, the second binding agent is selected from the group consisting of antibodies, ligands, receptors, fusion proteins, subunits of proteins, recombinant proteins, and fragments of the same. In particular embodiments, the second binding agent is an antibody and fragments of the same. In more particular embodiments, the antibody is a F(ab′)2 fragment, and is even more particularly an F(ab′)2 fragment that specifically binds to the Fc portion of an antibody.

In still other embodiments, the detectable label is a fluorophore, chemical dye, radioactive binding agent, chemiluminescent binding agent, electrochemiluminescent agent, magnetic binding agent, paramagnetic binding agent, promagnetic binding agent, enzyme that yield a colored product, enzyme that yield a chemiluminescent product, and enzyme that yield a magnetic product. In very particular embodiments, the detectable label is ruthenium or multiple ruthenium labels.

In some embodiments, the reagent comprises a resin, which, in particular embodiments, has a third binding agent attached to it. In certain embodiments, the third binding agent is selected from the group consisting of antibodies, ligands, receptors, fusion proteins, subunits of proteins, recombinant proteins, and fragments of the same. While in particular embodiments, the third binding agent is Protein A or streptavidin. In some embodiments, the protein is eluted from the reagent using a vacuum. In many embodiments, the screening of the incubated cell lines utilizes an automated workstation.

In certain embodiments, the condition to be tested is cell growth media. In other embodiments, the condition to be tested is temperature. In still other embodiments, the condition to be tested is humidity. In still more embodiments, the condition to be tested is pressure. In yet more embodiments, the condition to be tested is oxygen pressure.

In yet another aspect, the invention provides a method for high-throughput screening of cell lines for protein production. The method includes a first step of incubating the cell lines in media. A sample from each cell line is placed onto a solid support, i.e., the solid support is brought into contact with each sample. It should be noted that the solid support has a first binding agent attached to its surface that binds to a protein in each sample. The first binding agent binds the protein in the cell sample. The protein is contacted with a second binding agent, which is operably linked to a detectable label, that binds to the protein. The level of protein expression is determined in each sample by detecting the label operably linked to the second binding agent bound to the protein. The cell lines are selected for protein production based on whether the cell line has a desired level of protein expression as compared to the average level of protein expression in the cell lines screened.

In some embodiments, the protein is an antibody, ligand, receptor, subunit of protein, fragment of protein, fusion protein, recombinant protein, and fragment of the same. In certain embodiments, the protein is an antibody, a recombinant antibody, or a F(ab′)2 fragment.

In other embodiments, the first binding agent is selected from the group consisting of antibodies, ligands, receptors, fusion proteins, subunits of proteins, recombinant proteins, and fragments of the same. In particular embodiments, the first binding agent is Protein A or streptavidin. In still other embodiments, the binding agents can be attached to a solid support such as beads, plates, and microarray chips. In many embodiments, the solid support comprises cellulose, sepharose, polyacrylamide, glass, or polystyrene.

In other embodiments, the second binding agent is selected from the group consisting of antibodies, ligands, receptors, fusion proteins, subunits of proteins, recombinant proteins, and fragments of the same. In particular embodiments, the second binding agent is an antibody and fragments of the same. In more particular embodiments, the antibody is a F(ab′)2 fragment, and is even more particularly an F(ab′)2 fragment that specifically binds to the Fc portion of an antibody.

In still other embodiments, the detectable label is a fluorophore, chemical dye, radioactive binding agent, chemiluminescent binding agent, electrochemiluminescent agent, magnetic binding agent, paramagnetic binding agent, promagnetic binding agent, enzyme that yield a colored product, enzyme that yield a chemiluminescent product, and enzyme that yield a magnetic product. In very particular embodiments, the detectable label is ruthenium or multiple ruthenium labels. In certain embodiments, the method further comprises a sialic acid assay.

### BRIEF DESCRIPTION OF THE FIGURES

The foregoing and other objects of the present invention, the various features thereof, as well as the invention itself may be more fully understood from the following description, when read together with the accompanying drawings in which:

FIG. 1 is a pictorial representation of a high-throughput protein expression assay showing the detection of an antibody in a cell sample.

FIG. 2 is a graphical representation of a high-throughput screening assay showing the signal-to-background ratio of using various F(ab′)₂ fragments conjugated to ruthenium.

FIG. 3 is a graphical representation of a high-throughput screening assay showing the signal-to-background ratio of using various F(ab′)₂ fragments conjugated to ruthenium.

FIG. 4 is a graphical representation of a plot showing the sensitivity of high-throughput screening assays detecting GP1bα, IL13 receptor, and TNFR fusion protein at different concentrations.

FIG. 5 is a graphical representation of a plot showing the sensitivity of high-throughput screening assays detecting anti-GDF8, anti-CD22, and anti-Lewis Y antibodies at different concentrations.

FIG. 6 is a graphical representation of a plot showing the assay time required for the high-throughput titer screening assay and a HPLC assay.

FIG. 7 is a graphical representation showing a comparison between the high-throughput screening assay and HPLC at determining the levels of TNFR fusion protein in samples.

FIG. 8 is a graphical representation showing the levels of expression in clones as determined by a high-throughput titer screening assay.

FIG. 9 is a graphical representation showing a comparison between the high-throughput screening assay and HPLC at determining the levels of anti-Lewis Y antibodies in samples.

FIG. 10 is a graphical representation showing a comparison between the high-throughput screening assay and HPLC at determining the levels of PSGL and GP1bα in samples.

FIG. 11 is a graphical representation showing a comparison between the high-throughput screening assay and HPLC at determining the levels of anti-Aβ in samples.

FIG. 12 is a graphical representation showing the percentage of high molecular weight protein found in samples purified using different purification procedures.

FIG. 13 is a graphical representation showing the percentage of high molecular weight protein found in samples purified using different purification procedures.

FIG. 14 is a graphical representation of a bar graph showing the amount of high molecular weight protein found in samples isolated from different cell lines grown in different conditions.

### DETAILED DESCRIPTION OF THE INVENTION

The patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

1.1. General

An embodiment of the present invention in part provides methods of screening cell lines for the capability to produce a protein-of-interest. The invention also describes processes for improved efficiency in the industrial-scale production of proteins for pharmaceutical and biological studies. In particular, the present invention allows for the efficient production of proteins that can be utilized in pharmaceutical treatments of diseases such as cancer, Alzheimer's Disease, and diabetes. Furthermore, embodiments of the invention provide a method for high-throughput screening of cell lines to determine the quantity and quality of a protein-of-interest produced by the cell lines.

Accordingly, one aspect of the invention provides a method of high-throughput screening of cell lines for protein expression. The method utilizes a first binding agent that binds to the protein-of-interest and a second binding agent that is operably linked to a detectable label that binds to the protein-of-interest. In some embodiments, the first binding agent is attached to a solid support such as a bead, magnetic bead, a plate, or a microarray chip. The method also uses a reagent that binds to the protein-of-interest, and allows for the protein to be purified from a sample derived from the cell line. In some embodiments of the invention, the protein is purified from the reagent by means of a vacuum drawing the eluted protein from the reagent and through a filter. In still other embodiments, the solution is allowed to flow through the reagent by gravity flow.

As used here, the term “therapeutic protein” is a protein or peptide that has a biological effect on a region in the body on which it acts or on a region of the body on which it remotely acts via intermediates. A therapeutic protein can be, for example, a secreted protein, such as, an antibody, an antigen-binding fragment of an antibody, a soluble receptor, a receptor fusion, a cytokine, a growth factor, an enzyme, or a clotting factor, as described in more detail herein below. The above list of proteins is merely exemplary in nature, and is not intended to be a limiting recitation. One of ordinary skill in the art will understand that any protein may be used in accordance with the present invention and will be able to select the particular protein to be produced based as needed.

As used in the specification, the terms polypeptide, protein and peptide are synonymous and are used interchangeably. Accordingly, as used herein, the size of a protein, peptide or polypeptide generally comprises more than 2 amino acids. For example, a protein, peptide or polypeptide can comprise from about 2 to about 20 amino acids, from about 20 to about 40 amino acids, from about 40 to about 100 amino acids, from about 100 amino acids to about 200 amino acids, from about 200 amino acids to about 300 amino acids, and so on. As used herein, an amino acid refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

As used herein, term “antibody” is used to refer to any antibody-like molecule that has an antigen binding region, and includes antibody fragments such as Fab′, Fab, F(ab′).sub.2, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Harlow and Lane, Antibodies: A Laboratory Manual, Cold Spring Harbor Laboratory, 1988; incorporated herein by reference). For example, an antibody can include at least one, and preferably two full-length heavy chains, and at least one, and preferably two light chains. The term “antibody” as used herein includes an antibody fragment or a variant molecule such as an antigen-binding fragment (e.g., an Fab, F(ab′)2, Fv, a single chain Fv fragment, a heavy chain fragment (e.g., a camelid VHH) and a binding domain-immunoglobulin fusion (e.g., SMIP™). The antibody can be a monoclonal or single-specificity antibody. The antibody can also be a human, humanized, chimeric, CDR-grafted, or in vitro generated antibody. In yet other embodiments, the antibody has a heavy chain constant region chosen from, e.g., IgG1, IgG2, IgG3, or IgG4. In another embodiment, the antibody has a light chain chosen from, e.g., kappa or lambda. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). Typically, the antibody specifically binds to a predetermined antigen, e.g., an antigen associated with a disorder, e.g., a neurodegenerative, metabolic, inflammatory, autoimmune and/or a malignant disorder.

Small Modular ImmunoPharmaceuticals (SMIP™) provide an example of a variant molecule comprising a binding domain polypeptide. SMIPs and their uses and applications are disclosed in, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one aspect of the invention, a single domain antibody as used herein is a naturally occurring single domain antibody known as heavy chain antibody devoid of light chains. Such single domain antibodies are disclosed in WO 9404678 for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain, e.g., a VHH domain; (vii) a single chain Fv (scFv); (viii) a bispecific antibody; and (ix) one or more fragments of an immunoglobulin molecule fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; Huston et al. (1988) Proc. Natl. Acad. Sci. U.S.A. 85:5879-83). Such single chain antibodies are also intended to be encompassed within the term “antigen-binding fragment” of an antibody. These antibody fragments are obtained using conventional techniques known to those skilled in the art, and the fragments are evaluated for function in the same manner as are intact antibodies.

Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

As used herein, the term “cell line” means a cell that is maintained in culture and has acquired the ability to grow in ex vivo conditions. Cell lines can be either immortalized or transiently established as “primary cell lines.” In certain embodiments, cell lines are established by techniques known in the art (see, e.g., Kwak et al. (2006) Anim. Biotechnol. 17(1): 51-8). In some embodiments, the cell lines are antibody-producing cells, which can be produced by techniques known in the art (see, e.g., Dessain et al. (2004) J. Immunol. Methods. 291(1-2): 109-22.). The cell lines can also be obtained from commercial sources such as ATCC cell biology collections (American Type Culture Collections, Mannassas, Va.).

As used herein, the term “binding agent” means a molecule that can associate with any other molecule by way of covalent bonds, hydrogen bonds, ionic bonds, Van der Waals forces, London forces, or any combination of the forces. Binding agents include, but are not limited to, proteins and fragments thereof, peptidomimetic compounds, antibodies and fragments thereof, nucleic acids, toxins, and small molecules.

Binding agents can be disposed on a derivatized solid support through methods well known in the art. Solid supports include, but are not limited to, beads, magnetic beads, microarray chips, nitrocellulose membranes, nylon membranes, multiwell plates, and PVDF membranes. In some embodiments, the solid support is a plate in which an electrode is disposed beneath the plate, which creates a magnetic field that attracts a binding agent bound to a magnetic material. The plates are used in accordance with manufacturer's protocols (see Meso Scale Discovery, Gaithersburg, Md.).

Solid supports can be composed of glass, polystyrene, plastic, magnetic metals such as iron, polyacrylamide, sepharose, cellulose, or any inert support that does not affect the binding agents ability to bind a protein. Solid supports are obtained commercially from, e.g., Applied Biosystems, (Foster City, Calif.).

Moreover, in some embodiments, binding agents are disposed on a solid support such as a microarray chip utilizing methods practiced by those of ordinary skill in the art through a process called “printing” (see, e.g., Schena et. al., (1995) Science, 270(5235): 467-470). The term “printing,” as used herein, refers to the placement of spots onto the solid support in such close proximity as to allow a maximum number of spots to be disposed onto a solid support. The printing process can be carried out by, e.g., a robotic printer. The VersArray CHIP Writer Prosystem (BioRad Laboratories) using Stealth Micro Spotting Pins (Telechem International, Inc, Sunnyvale, Calif.) is a non-limiting example of a chip-printing device that can be used to produce the focused microarray for this aspect.

As used herein, the term “appropriate quality” means the quality that is particularly related to the protein-of-interest. An appropriate quality includes, but is not limited to, enzymatic reactions, antibody-epitope interactions, and nucleic acid-protein interactions. An appropriate quality can be assayed by analyzing a particular physicochemical aspect of the protein-of-interest. For instance, appropriate quality can be, without intending to limit the types of assays that can be used, related to the size, charge, carbohydrate content of the protein, binding activity, and enzymatic activity. Physical structure analyses such as NMR can be used to determine the overall tertiary and secondary structure of the protein. In addition, lectin-based assays and sialic acid assays, which will be described more fully below, can be used to determine the physicochemical aspects of the protein-of-interest. In other words, appropriate quality can be determined by looking not only to the chemical activities of the protein-of-interest, but to the physical structure of the protein-of-interest as well.

As used herein, the term “elute” means to extract from a resin or binding agent by the use of a solvent. The process of eluting a protein-of-interest from a resin or binding agent can involve a solution that contains a molecule capable of dislodging the protein-of-interest from the binding agent. In addition, the solution can have a pH that alters the binding characteristics of the resin or binding agent such that the protein-of-interest no longer associates with the protein-of-interest. It should be noted that any solution can be used to elute a protein in the present invention so long as the process does not affect the overall functionality or quality of the protein-of-interest.

As used herein, the term “resin” means any solid or semi-solid organic products of natural or synthetic origin. Resins include any material that can be conjugated to a moiety that allows for purification of a molecule from a complex mixture. Moieties useful in the present invention include cationic molecules, anionic molecules, metals, metalloids, polysaccharides, polypeptides, proteins, nucleic acids, peptides, small organic molecules, and peptidomimetic compounds. In particular, resins can themselves be composed of any inert compound including, but not limited to, sephadex, polystyrene, polyacrylamide, or neutral polysaccharides. Resins can be commercially obtained from, e.g., Clontech Laboratories, Inc. (Mountain View, Calif.).

The term “high-throughput” as used herein means allowing for a fast and simple methodology to determine the presence of desirable protein expression in a cell line. Desirable protein expression refers to both the quantity and quality of the biological molecules expressed by the cell lines screened by high-throughput techniques. High-throughput methodology also can include automated systems for processing the biological molecules and automated data processing for large-scale screening.

As used herein, the term “high-throughput titer screening” means a procedure used to determine the quantity of protein expressed by a cell. High-throughput titer screening includes the use of binding agents to identify a protein-of-interest in a sample derived from a cell. The binding agents can be operably linked to a detectable label or a solid support, all of which will be described more fully below.

As used herein, the term “high-throughput purification” means the process of purifying proteins from multiple cell samples at the same or substantially same time.

As used herein, the term “cell culture process development” means procedures to bring about the optimization of the conditions necessary to produce proteins at sufficient quantities and qualities for industrial or small-scale production. The conditions that can be tested using cell culture process development include, but are not limited to, salt concentration, media content, growth temperature, atmospheric pressure, atmospheric oxygen content, culture agitation, and carbon dioxide content. The cell culture process development includes the steps of determining the quantity of a protein (high-throughput titer screening) and the quality of a protein (high-throughput purification).

Methods of determining the quality of a protein for the purposes of cell culture process development include, but are not limited to, binding assays, lectin assays, sialic acid assays, NMR, circular dichroism, mass spectrometry, MALDI-TOF, enzymatic assays, colorimetric assays, and amino acid sequencing. These assays can be used at any time during the cell culture process development method. In certain embodiments, the assays are performed after the completion of the high-throughput purification of the protein-of-interest.

The term “protein-of-interest” as used herein means any protein, or protein-like molecule, produced for biological, medical, medicinal, or pharmaceutical purposes. For example, a protein-of-interest can be a therapeutic protein. Proteins-of-interest can be produced from nucleic acid sequences, whether chromosomal or extrachromosomal, which include, but are not limited to, pre-messenger RNA, messenger RNA, transfer RNA, heteronuclear RNA (“HnRNA”), ribosomal RNA, single-stranded DNA, and double-stranded RNA. Extrachromosomal sources of nucleic acid sequences can include double-strand DNA viral genomes, single-stranded DNA viral genomes, double-stranded RNA viral genomes, single-stranded RNA viral genomes, bacterial DNA, mitochondrial genomic DNA, cDNA or any other foreign source of nucleic acid that is capable of generating a protein-of-interest. A protein-of-interest can be any structure or combination of structures. For example, proteins-of-interest include, but are not limited to, recombinant proteins, proteins containing a quaternary structure, glycosylated proteins, lipidated proteins, oligopeptides, peptides, protein domains, protein subunits, antibodies or fragments thereof, and antibody-like molecules. Proteins-of-interest also include, for example, fusion proteins. Fusion proteins generally have all or a substantial portion of a targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. A fusion protein can include a targeting moiety, e.g., a soluble receptor fragment or a ligand, and an immunoglobulin chain, an Fc fragment, a heavy chain constant regions of the various isotypes, including: IgG1, IgG2, IgG3, IgG4, IgM, IgA1, IgA2, IgD, and IgE). For example, the fusion protein can include the extracellular domain of a receptor, and, e.g., fused to, a human immunoglobulin Fc chain (e.g., human IgG, e.g., human IgG1 or human IgG4, or a mutated form thereof). In one embodiment, the human Fc sequence has been mutated at one or more amino acids, e.g., mutated at residues 254 and 257 from the wild type sequence to reduce Fc receptor binding. The fusion proteins may additionally include a linker sequence joining the first moiety to the second moiety, e.g., the immunoglobulin fragment. For example, the fusion protein can include a peptide linker, e.g., a peptide linker of about 4 to 20, more preferably, 5 to 10, amino acids in length; the peptide linker is 8 amino acids in length. For example, the fusion protein can include a peptide linker having the formula (Ser-Gly-Gly-Gly-Gly)y wherein y is 1, 2, 3, 4, 5, 6, 7, or 8. In other embodiments, additional amino acid sequences can be added to the N- or C-terminus of the fusion protein to facilitate expression, steric flexibility, detection and/or isolation or purification.

Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

In certain embodiments, a fusion protein is a tumor necrosis factor inhibitor, for example in the form of tumor necrosis factor alpha and beta receptors (TNFR-1; EP 417,563 published Mar. 20, 1991; and TNFR-2, EP 417,014 published Mar. 20, 1991, each of which is incorporated herein by reference in its entirety), and is analysed in accordance with the present invention (for review, see Naismith and Sprang, J. Inflamm. 47(1-2):1-7, 1995-96, incorporated herein by reference in its entirety). According to some embodiments, a tumor necrosis factor inhibitor comprises a soluble TNF receptor. In certain embodiments, a tumor necrosis factor inhibitor comprises a soluble TNFR fused to any portion of an immunoglobulin protein, including the Fc region of an immunoglobulin. In certain embodiments, TNF inhibitors of the present invention are soluble forms of TNFR I and TNFR II. In certain embodiments, TNF inhibitors of the present invention are soluble TNF binding proteins. In certain embodiments, the TNF inhibitors of the present invention are TNFR-Fc, for example, etanercept. As used herein, “etanercept,” refers to a TNFR-Fc, which is a dimer of two molecules of the extracellular portion of the p75 TNF-α receptor, each molecule consisting of a 235 amino acid Fc portion of human IgG1. In accordance with the invention, an anti-senescence compound, such as carnosine, is used to decrease the amount of misfolded and/or aggregated protein during the production of TNFR-Fc.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The coding regions for known genes may be amplified and/or expressed using the techniques disclosed herein or as would be know to those of ordinary skill in the art (see, e.g., Kaleeba et al. (2006) Science 311 (5769):1921-4). Alternatively, various commercial preparations of proteins, polypeptides and peptides are known to those of skill in the art.

Furthermore, as used herein, the term “desired level of expression” means the quantity of protein necessary, depending on the physicochemical characteristics of the protein, to allow for subsequent purification of the protein. The desired levels of expression of a protein-of-interest are dependent on a number of factors related to the production methods to be utilized. For instance, the level of expression from a particular cell line allows for protein quality and quantity analysis as well as efficient purification of the protein. The desired level of protein expression from a cell line, therefore, is determined by one of skill in the art in view of the protein's characteristics and the purification and assay methods to be used on the protein.

In certain embodiments, the desired levels of protein expression are selected based on the particular requirements for the protein-of-interest. For instance, one of skill in the art can choose a desired level of protein expression to be an increased level of expression of a protein-of-interest to maximize the amount of protein produced. In other embodiments, a decreased level of expression of the protein-of-interest is chosen to be the desired level of protein expression in the case of proteins, such as a toxin, that are toxic at high levels in the cell producing the toxin. In addition, a decreased level of protein expression is selected in situations when the protein-of-interest forms inclusion bodies at high concentration levels. Therefore, the level of protein expression selected by one of skill in the art depends on the characteristics of the protein-of-interest.

In determining the quantity and quality of a protein produced by a cell line, a cell line sample is typically necessary for assessment of protein expression and protein quality. In certain embodiments, a cell line sample is isolated using means that are known in the art such as cell lysis and supernatant isolation (see, e.g., Vara et al. (2005) Biomaterials 26(18): 3987-93; Iyer et al. (1998) J. Biol. Chem. 273(5):2692-7). Alternatively, cell line samples are isolated from a medium that has a secreted protein such as an antibody, extracellular matrix protein, or serum protein. In such embodiments, the medium is the sample that will be tested for protein quantity and quality. In one embodiment, the medium sample is used in an assay to test for protein quantity, in the absence of any prior purification steps, as further described herein.

Another aspect of the invention provides a method of high-throughput screening of cell lines for protein production. In this method, the levels of protein expression are determined by contacting a solid support with a cell line sample containing a protein. In one embodiment, the cell line sample is cell culture media. The solid support has attached to its surface a first binding agent that is capable of binding to the protein. The method also includes a second binding agent that binds to the protein bound by the first binding agent and immobilized on the solid support. The second binding agent is operably linked to a detectable label. The cell lines are selected based on the desired level of expression required for the protein.

The level of expression of a particular protein can be measured by “dot blot” in which a first binding agent is immobilized on a membrane such as nitrocellulose, nylon, or PVDF (see, e.g., Heinicke et al. (1992) J. Immunol. Methods. 152(2): 227-36.). Protein microarray technology can also be used to determine the expression of proteins in a sample. Alternatively, a sample is placed into a well of a multiwell plate that contains the first binding agent. In such embodiments, similar techniques such as ELISA analysis are routine in the art (see, e.g., Ausubel, et al. (1996) Current Protocols in Molecular Biology, Vol. 1, pp. 4.2.1-4.2.9, John Wiley & Sons, Inc.

In some embodiments, the high-throughput screening and/or purification of the cell line samples are performed using an automated workstation. In addition, the cell culture process development can be performed using automated workstations. Automated workstations are commonly utilized in the art to perform many experiments in a short period of time. Examples of automated workstations include, but are not limited to, the TECAN Genesis Workstation (TECAN Schweiz AG, Mannedorf, CH) and the Biomek FX Workstation (Beckman Coulter, Fullerton, Calif.). Methods of use can be obtained from the manufacturers of automated workstations and are well known in the art.

1.2. Binding Agents

Aspects of the invention utilize binding agents to bind a protein-of-interest. In certain embodiments, a binding agent is an antibody or fragment thereof. Where the binding agent that specifically binds a protein is an antibody, the antibody may be, without limitation, a polyclonal antibody, a monoclonal antibody, a chimeric antibody, a humanized antibody, a genetically engineered antibody, a bispecific antibody (where one of the specificities of the bispecific antibody specifically binds to the triosephosphate isomerase protein), antibody fragments (including but not limited to “Fv,” “F(ab′)₂,” “F(ab),” and “Dab”); and single chains representing the reactive portion of an antibody (“SC-MAb”). Methods for making antibodies and other binding agents are well known (see, e.g., Coligan et al. (1991) Current Protocols in Immunology, John Wiley and Sons, Inc.; Jones et al. (1986) Nature 321: 522-525; Marx (1985) Science 229: 455-456; Rodwell (1989) Nature 342: 99-100; Clackson (1991) Br. J. Rheumatol. 3052: 36-39; Reichman et al. (1988) Nature 332: 323-327; Verhoeyen, et al. (1988) Science 239: 1534-1536).

The binding agent can be an antibody or fragment thereof that binds to the Fc portion of an antibody. In certain embodiments, the binding agent allows for detection of antibodies in a cell line sample. In particular embodiments, an antibody, which is the second binding agent, is detectably labeled with an electrochemiluminescent detectable label such as ruthenium. In addition, the second binding agent can be a F(ab′)₂ fragment detectably labeled with an electrochemiluminescent detectable label such as ruthenium.

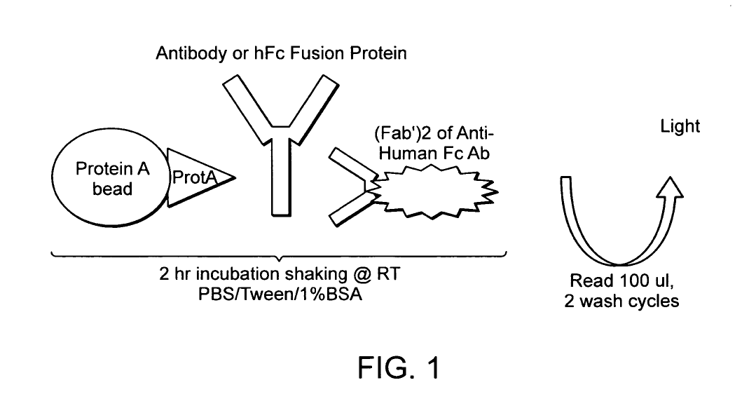

The detection of a protein of interest using F(ab′)₂ fragments is shown in FIG. 1. The F(ab′)₂ fragment is operably linked to a detectable label such as an Ori-Tag. In FIG. 1, the F(ab′)2 fragment recognizes the Fc portion of an antibody, which is a protein-of-interest in this pictorial example. The antibody has been immobilized on a bead, which has Protein A or streptavidin conjugated to it (FIG. 1). The binding of the F(ab′)₂ fragment is observed as a generation of light (FIG. 1).

It is important to note that the antibodies used as a first binding agent in an aspect of the present invention can be coupled to the surface of a solid support. Coupling of the first binding agent improves the signal strength of the reaction and produces improved results. Common coupling agents include, but are not limited to, silanization using (3-mercaptopropyl) trimethoxysilane, agarose coating, and poly-L-lysine films. Additionally, recombinant antibodies can be engineered to include a tag facilitating coupling to the support. For example, a recombinant antibody having a histidine tag can be coupled to supports coated with nickel.

In addition, compounds such as peptides, peptidomimetic compounds, and small molecules can be used as binding agents. Binding agents can be synthesized from peptides or other biomolecules, including, but not limited, to saccharides, fatty acids, sterols, isoprenoids, purines, pyrimidines, derivatives or structural analogs of the above, or combinations thereof and the like. Phage display libraries and chemical combinatorial libraries can be used to develop and select synthetic compounds that are acceptable binding agents for a protein-of-interest. Also envisioned in the invention is the use of potential binding agents made from peptoids, random bio-oligomers (U.S. Pat. No. 5,650,489), benzodiazepines, diversomeres such as dydantoins, benzodiazepines and dipeptides, nonpeptidal peptidomimetics with a beta-D-glucose scaffolding, oligocarbamates or peptidyl phosphonates.

In certain examples, binding agents can be peptides that are designed to specifically interact, bind, or associate with a protein. Peptide binding agents can also interact, associate, or bind with an amino acid sequence of any other protein. Peptides can be subjected to directed or random chemical modifications such as acylation, alkylation, esterification, amidification, etc.

Identification and screening of peptide binding agents is further facilitated by determining structural features of the protein-of-interest, e.g., using X-ray crystallography, neutron diffraction, nuclear magnetic resonance spectrometry, and other techniques for structure determination. Computer algorithms can further facilitate binding agent identification. Such computer algorithms are employed that are capable of scanning a database of peptides and small molecules of known three-dimensional structure for candidates that fit geometrically into the target protein's site (see, e.g., Chen and Kellogg (2005) J. Comput. Aided Mol. Des. 19(2):69-82). Most algorithms of this type provide a method for finding a wide assortment of chemical structures that are complementary to the shape of a binding pocket or region of a domain of a protein. Each of a set of peptides from a particular database can be compared to determine the particular peptides that have the most potential for interacting with a protein-of-interest.

The compounds of the present invention can also be peptidomimetic compounds that can be at least partially unnatural. The peptidomimetic compound can be a small molecule mimic of a portion of any desirable amino acid sequence. The compound can have increased stability, efficacy, potency and bioavailability by virtue of the mimic. Further, the compound can have decreased toxicity. The peptidomimetic compound can have enhanced mucosal intestinal permeability. The compound can be synthetically prepared. The compound of the present invention can include L-,D- or unnatural amino acids, alpha, alpha-disubstituted amino acids, N-alkyl amino acids, lactic acid (an isoelectronic analog of alanine). The peptide backbone of the compound can have at least one bond replaced with PSI-[CH═CH] (Kempf et al. (1991) Int. J. Pept. Protein Res. 38(3): 237-41). The compound can further include trifluorotyrosine, p-Cl-phenylalanine, p-Br-phenylalanine, poly-L-propargylglycine, poly-D,L-allyl glycine, or poly-L-allyl glycine.

One example of the present invention is a peptidomimetic compound wherein the compound has a bond, a peptide backbone or an amino acid component replaced with a suitable mimic. Examples of unnatural amino acids which can be suitable amino acid mimics include, but are not limited to, β-alanine, L-α-amino butyric acid, L-γ-amino butyric acid, L-α-amino isobutyric acid, L-ε-amino caproic acid, 7-amino heptanoic acid, L-aspartic acid, L-glutamic acid, cysteine (acetamindomethyl), N-ε-Boc-N-α-CBZ-L-lysine, N-ε-Boc-N-α-Fmoc-L-lysine, L-methionine sulfone, L-norleucine, L-norvaline, N-α-Boc-N-δ-CBZ-L-ornithine, N-δ-Boc-N-α-CBZ-L-ornithine, Boc-p-nitro-L-phenylalanine, Boc-hydroxyproline, Boc-L-thioproline. (Blondelle, et al. (1994) Antimicrob. Agents Chemother. 38(10): 2280-6; Pinilla, et al. (1995) Biopolymers. 37(3): 221-40).

Sometimes, the binding agents can be small molecules that bind, interact, or associate with a protein. Such a small molecule can be an organic molecule that is capable of penetrating the lipid bilayer of a cell. Small molecules include, but are not limited to, toxins, chelating agents, metals, and metalloid compounds. Small molecules can be attached or conjugated to a targeting agent so as to specifically guide the small molecule to a particular cell.

In some embodiments, a binding agent is a nucleic acid sequence, which can be a full-length sequence, fragments of full-length sequences or synthesized oligonucleotides that bind under physiological conditions to a protein such as a transcription factor. “Nucleic acid” refers to a polymer comprising two or more nucleotides and includes single-, double-, and triple-stranded polymers. “Nucleotide” refers to both naturally occurring and non-naturally occurring compounds and comprises a heterocyclic base, a sugar, and a linking group, such as a phosphate ester. For example, structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

The binding agents of the present invention can be conjugated to a detectable label. According to the invention, a “detectable label” is a moiety that can be sensed. In some embodiments, a detectable label is operably linked to a binding agent. By “operably linked,” it is meant that the detectable label is attached to the binding agent by either a covalent or non-covalent (e.g., ionic) bond. Methods for creating covalent bonds are known (see general protocols in, e.g., Wong, S. S. (1991) Chemistry of protein Conjugation and Cross-Linking, CRC Press; Burkhart et al. (1999) The Chemistry and Application of Amino Crosslinking Agents or Aminoplasts, John Wiley & Sons Inc.).

In accordance with the invention, a detectably labeled binding agent includes a binding agent that is conjugated to a detectable moiety. Another detectably labeled binding agent of the invention is a fusion protein, where one partner is the binding agent and the other partner is a detectably label. Yet a further non-limiting example of a detectably labeled binding agent is a first fusion protein comprising a binding agent and a first moiety with high affinity a second moiety, and a second fusion protein comprising a second moiety and a detectable label. For example, a binding agent that specifically binds to a protein is operably linked to a streptavidin moiety. A second fusion protein comprising a biotin moiety operably linked to a fluorescein moiety is added to the binding agent-streptavidin fusion protein, where the combination of the second fusion protein to the binding agent-streptavidin fusion protein results in a detectably labeled binding agent (i.e., a binding agent operably linked to a detectable label). In particular embodiments, the detectable label is detectable by a medical imaging device or system. For example, where the medical imaging system is an X-ray machine, the detectable label that can be detected by the X-ray machine is a radioactive label (e.g., ³²P). Note that a binding agent need not be directly conjugated to the detectable moiety. For example, a binding agent (e.g., an antibody) that is itself specifically bound to by a secondary detectable binding agent (e.g., a FITC labeled goat anti-mouse secondary antibody) is operably linked to a detectable moiety (i.e., the FITC moiety).

Detectable labels can be, without limitation, fluorophores (e.g., fluorescein (FITC), phycoerythrin, rhodamine), chemical dyes, or compounds that are radioactive, chemoluminescent, electrochemiluminescent, magnetic, paramagnetic, promagnetic, or enzymes that yield a product that may be colored, chemoluminescent, or magnetic. The signal is detectable by any suitable means, including spectroscopic, photochemical, biochemical, immunochemical, electrical, optical or chemical means. In certain cases, the signal is detectable by two or more means. In certain embodiments, protein labels include fluorescent dyes, radiolabels, electrochemiluminescent, and chemiluminescent labels.

For example, amino acids of binding agents may be conjugated to Cy5/Cy3 fluorescent dyes. These dyes are frequently used in the art (see, e.g., Linder et al. (2002) Electrophoresis. 23(5): 740-9). The fluorescent labels can be selected from a variety of structural classes, including the non-limiting examples such as 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

Other useful dyes are chemiluminescent dyes and can include, without limitation, biotin conjugated amino acids. In particular embodiments, electrochemiluminescent probes are conjugated to binding agents. As used herein, “electrochemiluminescence” is a chemiluminescent reaction that occurs subsequently to an electrochemical reaction. Electrochemiluminescent probes include, but are not limited to, luminol, acridan ester, ruthenium, ruthenium chelate, and ruthenium tribipyridine, NHS ester. Electrochemiluminescent probes can be obtained commercially from, e.g., BioVeris Corp. (Gaithersburg, Md.).

1.3 Analytical Assays

Aspects of the present invention also utilize assays of a protein's quality. These assays can be utilized during high-throughput titer screening of the protein-of-interest. Protein quality can also be determined during cell culture process development. As part of protein quality, protein structure includes the primary, secondary, tertiary, and quaternary structure of a protein as well as post-translational modifications such as glycosylation, lipidation, and phosphorylation. In addition, the size, shape, and charge of the protein affect the quality of the protein. The physical structure of a protein has a significant effect on the ability of a protein to perform its normal functions. In the context of enzymatic reactions, protein structure is vitally important for full enzymatic quality. In the context of antibodies or fragments thereof, the size, shape, surface charge, glycosylation, and phosphorylation of amino acids of the antibody has a significant effect on the antibody's epitope specificity.

In some embodiments, NMR, Matrix assisted laser desorption/time-of-flight (“MALDI-TOF”) analysis, and circular dichroism are used to determine the physical structure of a protein (see, e.g., U.S. Pat. Nos. 6,930,305; 7,005,272; and 7,029,872). Such techniques provide a detailed analysis of a protein's overall physical structure. These techniques are well known in the art.

In particular embodiments, size exclusion chromatography is used to determine the size of the protein of interest (see, e.g., Brooks et al. (2000) Proc. Natl. Acad. Sci. USA. 97(13): 7064-7067). In addition, cation exchange chromatography can be used to determine the charge of a protein (see, e.g., Zhang and Glatz (1999) Biotechnol. Prog. 15(1): 12-18). Other techniques that can be utilized to identify the structure of a protein-of-interest include, but are not limited to, reverse phase HPLC, capillary electrophoresis SDS, capillary zone electrophoresis, and high pH anionic exchange HPLC. These techniques can be practiced using procedures that are well known in the art.

Other structural assays include the sialic acid assay and the lectin assay. These assays identify the level of glycosylation found on the surface of a protein, which informs on the quality of the protein. Sialic acid assays have been used to determine the extent of carbohydrates present in a sample, and these techniques are known in the art (see, e.g., U.S. Pat. Nos. 5,807,553 and 5,855,901). Lectin based assays also detect the presence of carbohydrate in a sample, but through a mechanism of a protein-carbohydrate interaction (see, e.g., U.S. Pat. No. 5,633,148). Lectin assays have been used extensively in the art for carbohydrate binding, and are described, for example, in U.S. Pat. No. 6,331,319.

In addition to determining the physical structure of a protein, the ability of a protein to perform certain functions can be assayed (see, e.g., U.S. Pat. No. 7,029,862). Also, the binding function of a protein or polypeptide (e.g., encoded by hybridizing nucleic acid) can be detected in binding or binding inhibition assays, using membrane fractions containing receptor or cells expressing receptor, for example (see e.g., Van Riper et al. (1993) J. Exp. Med., 177: 851 856; Sledziewski et al., U.S. Pat. No. 5,284,746). Thus, the ability of the encoded protein or polypeptide to bind a ligand, an inhibitor and/or promoter, can be assessed. The antigenic properties of proteins or polypeptides encoded by nucleic acids of the present invention can be determined by immunological methods employing antibodies that bind to a protein, such as immunoblotting, immunoprecipitation and immunoassay (e.g., radioimmunoassay, ELISA).

The signaling function of a protein or polypeptide (e.g., encoded by hybridizing nucleic acid) can be detected by enzymatic assays. The stimulatory function of a protein or polypeptide (e.g., encoded by hybridizing nucleic acid) can be detected by standard assays for chemotaxis or mediator release, using cells expressing the protein or polypeptide (e.g., assays which monitor chemotaxis, exocytosis (e.g., degranulation of enzymes, such as esterases (e.g., serine esterases), perforin, granzymes) or mediator release (e.g., histamine, leukotriene) in response to a ligand or a promoter (see e.g., Taub et al. (1995) J. Immunol., 155: 3877-3888; Baggliolini, M. and C. A. Dahinden (1994) Immunology Today, 15: 127-133 and references cited therein). Functions characteristic of a protein receptor can also be assessed by other suitable methods.

To demonstrate the methods according to the invention, screening methods as described above were performed on various cell lines for the purpose of identifying those cell lines that produced both a sufficient quantity of protein and sufficient quality of protein.

### EXAMPLES

Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Human Fc Protein Assay High-throughput Screening

1. Preparation of Anti-Aβ Standards

A Standard Curve Buffer (SCB) was prepared by mixing 20 ul of Media R5CD1, and 24 ml Assay Buffer (PBS w/0.05% Tween 20 and 1% bovine serum albumin) in 16 assay plates (Corning/Costar, Corning, N.Y.). A 0.3 mg/ml intermediate standard was prepared using 6 ul of 32.5 mg/ml reference standard and 644 ul SCB (prepared fresh each time). One ug/ml standard was placed into a well designated A1 and into an additional well designated H1 in a 2 ml deep-well plate. Well A1 contained 6 ul of 0.3 mg/ml intermediate standard and 1794 ul of SCB in 16 assay plates. This process was repeated for well H1. Serial dilutions were prepared by taking 900 ul of solution in the wells and adding 900 ul of SCB to each of the 16 assay plates. The dilutions were distributed 50 ul per standard well.

2. Preparation of Controls

Controls were prepared to produce a concentration of 120 ug/ml intermediate control. Briefly, 6 ul of 32.5 mg/ml reference standard and 1619 ul of Media R5CD1 were mixed. Dilutions of 1:40 were prepared by mixing 5 ul of 120 ug/ml intermediate control and 195 ul of Assay Buffer, while dilutions of 1:400 were prepared by mixing 20 ul of 1:40 diluted control and 180 ul of Assay Buffer. Dilutions of 1:1200 dilution were prepared by taking 80 ul of 1:400 diluted control and mixing in 160 ul Assay Buffer per every two assay plates. The 1:1200 dilutions were distributed in 50 ul amounts per 0.1 control well.

In addition, controls containing 0.01 μg/ml of control were prepared by mixing 150 ul of 120 ug/ml intermediate control in (prepared as above) in 1350 ul of Media. The 1:40 dilution was prepared by mixing 5 ul of 12 ug/ml intermediate control in 195 ul of Assay Buffer. The 1:400 dilution was prepared by mixing 20 ul of 1:40 diluted control in 180 ul of Assay Buffer and the 1:1200 dilution was prepared by mixing 80 ul of 1:400 diluted control in 160 ul Assay Buffer/2 assay plates. The controls were distributed in 50 ul aliquots of 1:1200 diluted control per 0.01 control well.

2. Preparation of ORI-Tagged F(ab′)₂ Fragment

Preparation of ORI-tagged Anti-Fc F(ab′)₂ fragment (Jackson ImmunoResearch Laboratories, Inc., West Grove, Pa.) was accomplished using the following protocol. Briefly, 50 μl DMSO was added to one vial of ORITAG NHS ester (BioVeris, Gaithersburg, Md.). The mixture was vortexed at the maximum setting until the ORITAG in the bottom of the vial dissolved. Then, 1638 μl of affiniPure F(ab′)2 Fragment Goat Anti-Human IgG antibody was added to 50 μl of dissolved ORITAG NHS ester to 1638 ul. The mixture was vortexed and incubated at room temperature for 60 minutes in a dark wrap. The vial was rotated on a rocker during the incubation.

The reaction was stopped by adding 20 μl of 2M glycine, and the tube was wrapped with foil and incubated for 10 minutes at room temperature. During incubation, two PD10 (Pharmacia, Piscataway, N.J.) columns with PBS were equilibrated with 0.1% NaN₃ and used according to manufacturer's protocol. The reaction tubes were centrifuged for 5 seconds to collect all of the volume in the tube. Then, 854 ul of the reaction was added to each PD10 columns. The samples were loaded to the columns and eventually 8 tubes of 0.5 ml aliquots were collected from each column.

The protein concentration was determined by BCA Protein Assay kit. In addition, the un-labeled leftover antibody from the second step was used as a standard to determine protein concentration. The absorbance of the protein samples was measured at 455 nm.

Fractions with appropriate protein concentrations and good ORI-TAG: Protein ratio were pooled. Bovine serum albumin was added to the final vial to make 1% BSA solutions. Vials were stored at 40° C.

3. Cell Line Sample Preparation and Reaction

A 1:40 dilution was prepared by mixing 5 ul of a sample from a cell line generating GP1bα, IL13R, anti-CD22 antibody, anti-Lewis Y antibody, anti-Aβ antibody, or TNFR fusion protein and 195 ul of Assay Buffer, while a 1:400 dilution of sample was prepared by mixing 20 ul of 1:40 diluted sample and 180 ul of Assay Buffer. Finally, a 1:1200 dilution was prepared by taking 70 ul of 1:400 diluted samples and mixing the samples with 140 ul Assay Buffer. This final dilution was distributed in 50 ul to each sample well. The 1:400 were also distributed to sample wells in another assay plate.

The tagged F(ab′)₂ fragment was distributed in aliquots of 5350 μl comprising 6 ul of F(ab′)₂ fragment in 5344 ul Buffer to each assay plate. There was 50 ul of solution per well.

Protein A beads were obtained from Dynal Biotech (Carlsbad, Calif.). Beads were distributed in 30 ul quantities in 5 ml of Assay buffer per plate. The solutions was distributed in 50 ul volumes per well.

All reagents and samples were distributed to the plates as follows: The load standard, control, and samples were loaded into the wells first. Then the anti-Fc ORI-tagged F(ab′)₂ fragment was loaded. Finally, the Protein A beads were loaded.

The mixture was incubated for 2 hours at room temperature with mixing, and read on an M8 or M384 analyzer with method “FcHuman150.”

4. Data Analysis

The Standard Acceptance Guidelines used for the experiments were at least 8 out of 10 standards the CV of the readings between standard point replicates should be about 20%. In addition, the Control Acceptance Guidelines had to be within 80 to 120%. Also, the Sample Acceptance Guidelines required a CV of the readings between sample replicates to be about 20%. Only the readings that fell within these ranges were taken into consideration.

5. Results

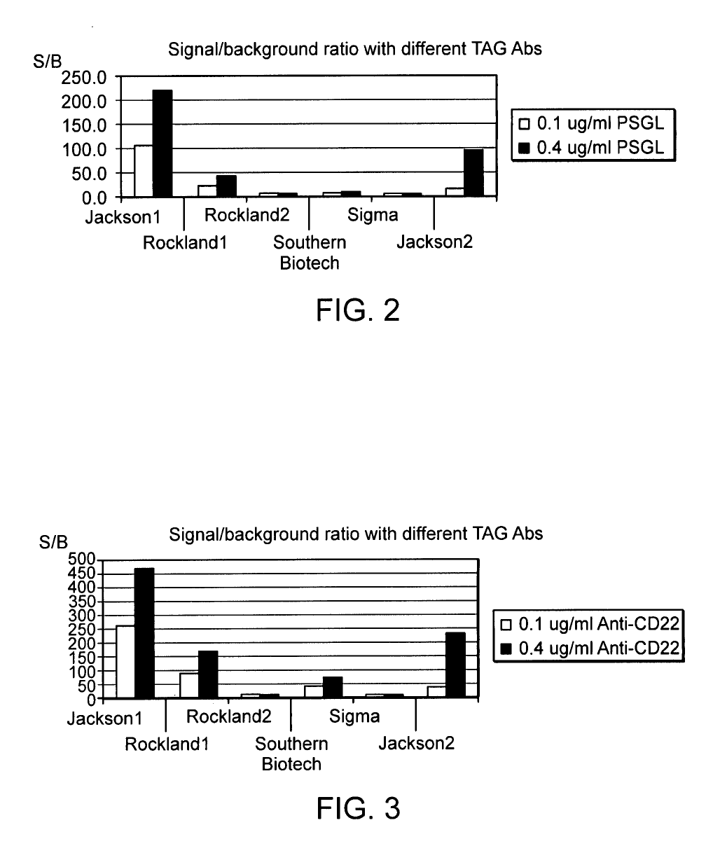

The labeling of the F(ab′)₂ fragment with ruthenium directed against PSGL showed significantly higher signal to noise ratios (FIG. 2). The data for 0.1 μg/ml and 0.4 μg/ml of F(ab′)₂ fragments were normalized against noise and placed onto the bar graph. The Jackson 1, Jackson 2, Rockland 1, and Southern Biotech F(ab′)₂ fragments had increased signal to noise ratios when labeled with ruthenium (FIG. 2). These experiments were confirmed using F(ab′)₂ fragments directed against anti-CD22 (FIG. 3).

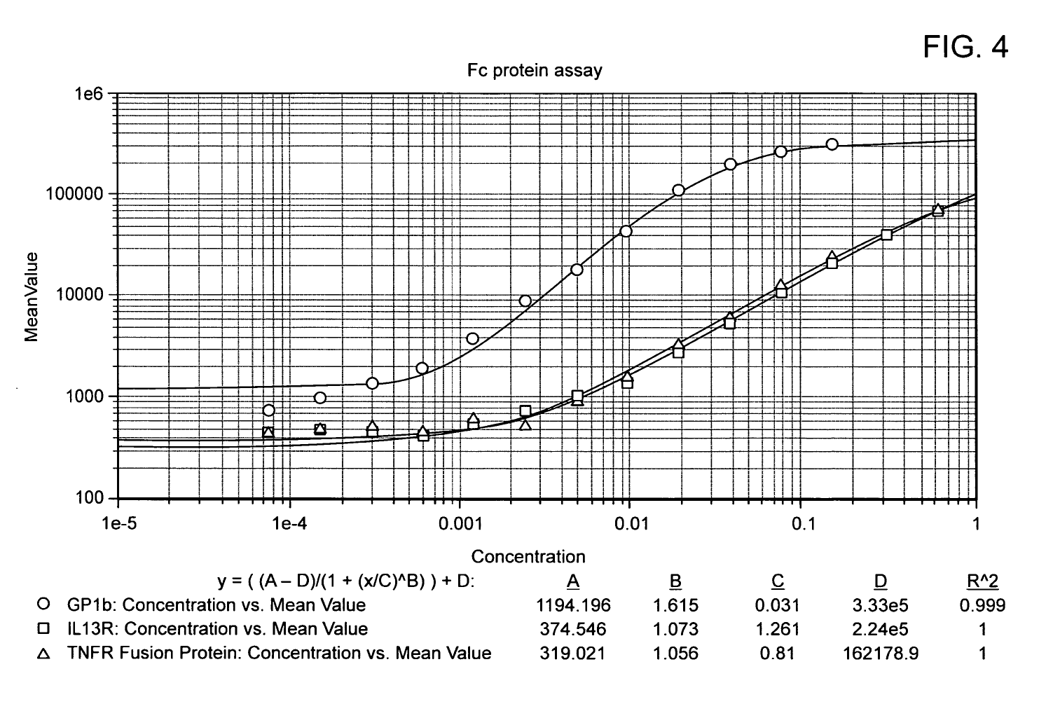

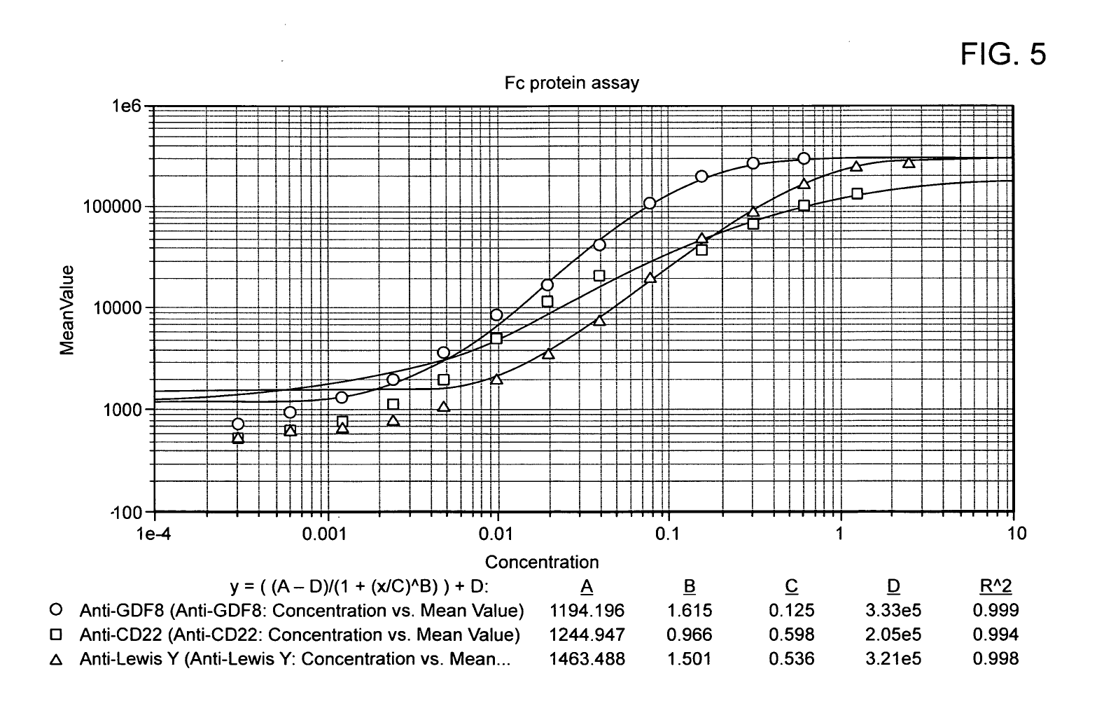

These results were further detailed in experiments utilizing F(ab′)₂ fragments directed against the Fc fusion proteins GP1bα, IL13 receptor, and TNFR fusion protein (FIG. 4). The plot shows that as the concentration of the target fusion proteins increased, the detection of the proteins with the F(ab′)₂ fragments increased (FIG. 4). Similar results were also obtained using anti-GDF8, anti-CD22, and anti-Lewis Y antibodies as the target rather than the Fc fusion proteins (FIG. 5).

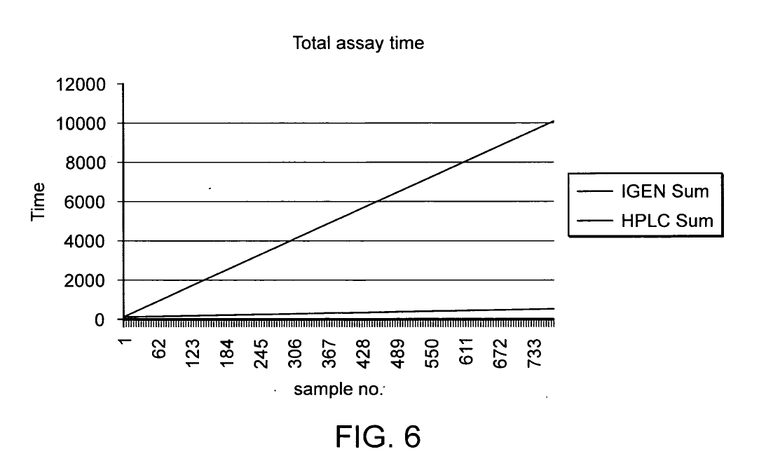

Using the labeled F(ab′)₂ fragments to identify the quantity of protein in a sample, the titer screening methodology was tested against standard column procedures such as HPLC. The results showed that the titer screening methodology was much faster rates than HPLC chromatography to determine the titer of the proteins (FIG. 6). The time required to obtain the protein quantity readings was more than 10 times greater using HPLC as compared to the high-throughput titer screening when up to over 700 samples were analyzed (FIG. 6).

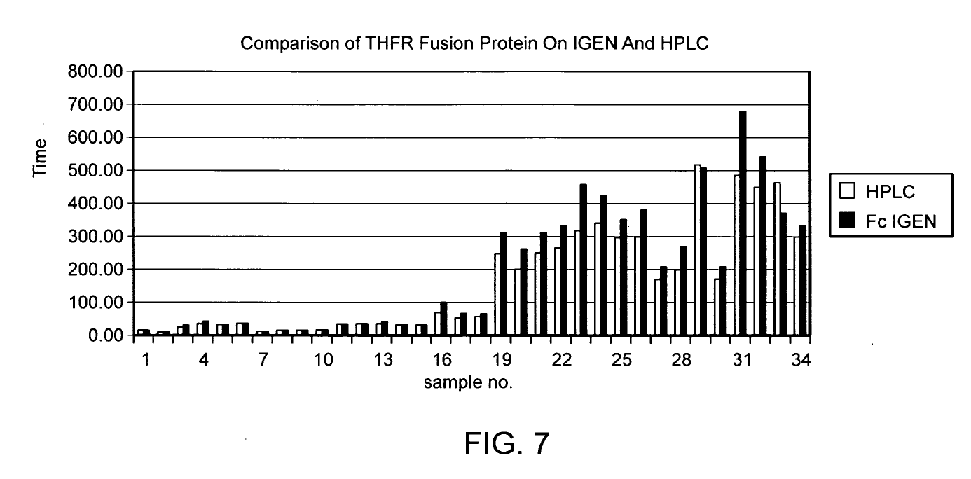

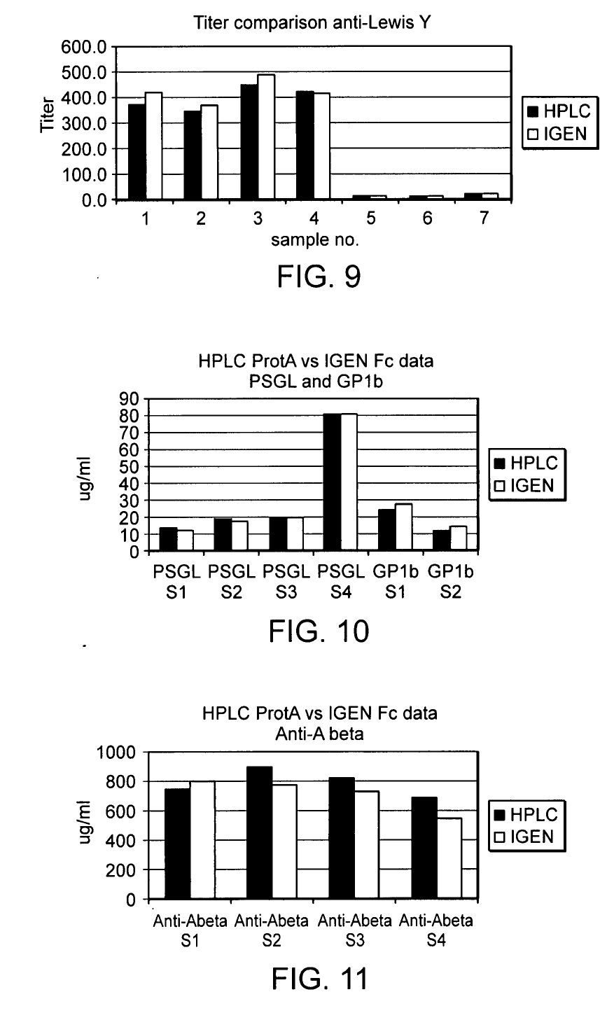

In addition to the high-throughput screening being faster than standard column procedures, it is as accurate in determining protein quantity (FIGS. 7, 9, 10, and 11). When HPLC and the titer screening procedures detailed above were compared, near identical titer quantities were identified for anti-Lewis Y protein, PSGL, anti-Aβ, and TNFR fusion protein (FIGS. 7, 9, 10, and 11). Accordingly, the titer screening assay detailed herein was faster and had a similar efficiency as the standard column techniques at determining protein quantity.

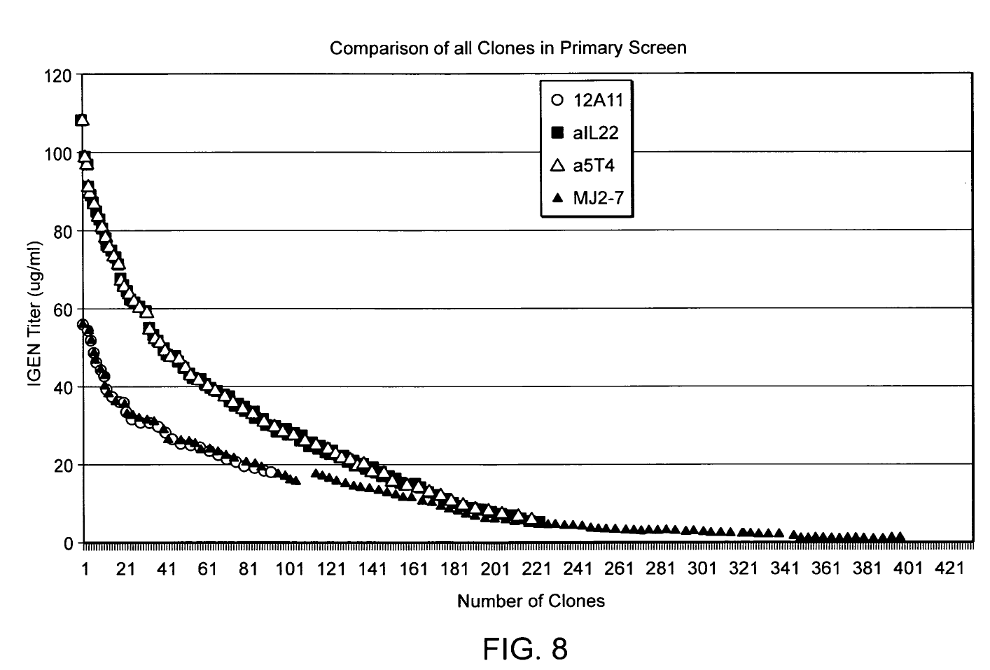

The high-throughput screen utilized above was able to identify cell lines that were expressers of the appropriate quantity of antibody (FIG. 8). As shown in FIG. 8, the high-throughput titer screening allowed for the identification of the highest producers of the antibody-of-interest, which is indicated by the increasing titer (μg/ml) per clone. In these experiments, the top titer clone is numbered 1, the second highest titer clone is numbered two, and so on. Therefore, the top clones were quickly identified by the assay.

### Example 2

### High-Throughput Purification and Identification of Proper Cell Culture Conditions

1. Manual Purification of Proteins Using Centrifugation

The high-throughput titer screening procedure of Example 1 can be linked to the high-throughput purification procedure detailed below to improve the potential development of cell culture development.

The thin resin came in 20% Ethanol. Additional 20% Ethanol solution was added to make up 50% of the settled volume. The solution was mixed thoroughly and dispensed in 200 uL aliquots per well of a filterplate (Whatman 7700-2804, long drip, 25 um, 96 well×800 uL, Whatman LabSciences, Orange, N.J.). The filterplates were stacked on top of empty microplates (Corning/Costar, Corning, N.Y.), and centrifuged (Sorvall Legend RT) for 3 min at 700 rpm (approximately equals 104 G) to remove the 20% Ethanol. Then, 200 uL per well of RODI water was added, and the plates were centrifuged for 3 min with empty microplates underneath. This process was repeated twice.

Wash buffer was added at 200 uL aliquots per well. The plates were centrifuged for 3 min with empty microplates underneath. This process was repeated two more times. When all samples were at least 170 ug/mL, the minimal dilution was made to make the titers of the same set of samples to be approximately the same range (+/−20%). The samples were diluted to be close to the lowest concentration. Any samples under 170 ug/mL were loaded more than once. When multiple loading was required, the samples were diluted only to ensure that the total mass of protein loaded was in the same range (+/−20%). When multiple loading was performed, the samples that required were added first and in the empty wells 200 uL of wash buffer was added. The filterplates were centrifuged as needed.

All samples were loaded together with reference material spikes and media blanks as usual. The samples were loaded onto the Protein A resin (Protein A Mab Select, Amersham Biosciences) at a load volume of 500 uL. A spiking standard was prepared in media with the concentration close to the whole set of test samples. The spiking material and media blank were loaded at 500 ul/well.

The resin was resuspended with sample by mixing with multi-channel pipette. The resin and sample were incubated for 5-10 minutes at room temperature. The mixture was then centrifuged at 700 rpm for 3 min., and the sample flow-through was collected in a deep well microplate (Whatman 7710-5750, Whatman LabSciences, Orange, N.J.). Then, 200 uL per well of wash buffer (5 mM Tris, 20, 50 or 100 mM NaCl, pH 7.5) was added and the mixture was centrifuged for 3 min with empty microplates underneath. Wash buffer was added at 200 uL per well. The samples were centrifuged for 3 min with empty microplates underneath. Then, 4 uL Tris (2.0 M Tris, pH 8.5, or 1.0 M Tris pH 8.5) was added to the mixture for neutralization to the UV collection plate (Corning/Costar, Corning, N.Y.) before performing elution. Elution buffer (50 mM Glycine, 20 or 50 or 100 mM NaCl, pH 2.5 or 3.0) was added at 200 uL volumes per well to filter plate. The resin and the elution buffer were mixed with a multi-channel pipette. The filter plate was then centrifuged for 3 min with the collection plate underneath. Plates were read at A280 on a Spectra Max plate reader (Molecular Devices).

2. Protein Quality Determinations Using Column Chromatography

For CEX assay: The elution was transferred at 50 uL aliquots into an Agilent plate containing 100 uL of CEX mobile phase A. The solution was mixed. Column purification procedures were performed using standard procedures.

For SEC assay: The elution was transferred at 130 uL volumes to an Agilent plate. The solution was mixed. Column purification procedures were performed using standard procedures.

For HIC and Sialic acid assay: Four microliters of 2M Tris was added to the UV plate and eluted directly into UV plate. The solution was mixed and read at A₂₈₀ on a Spectra Max plate reader.

3. Results

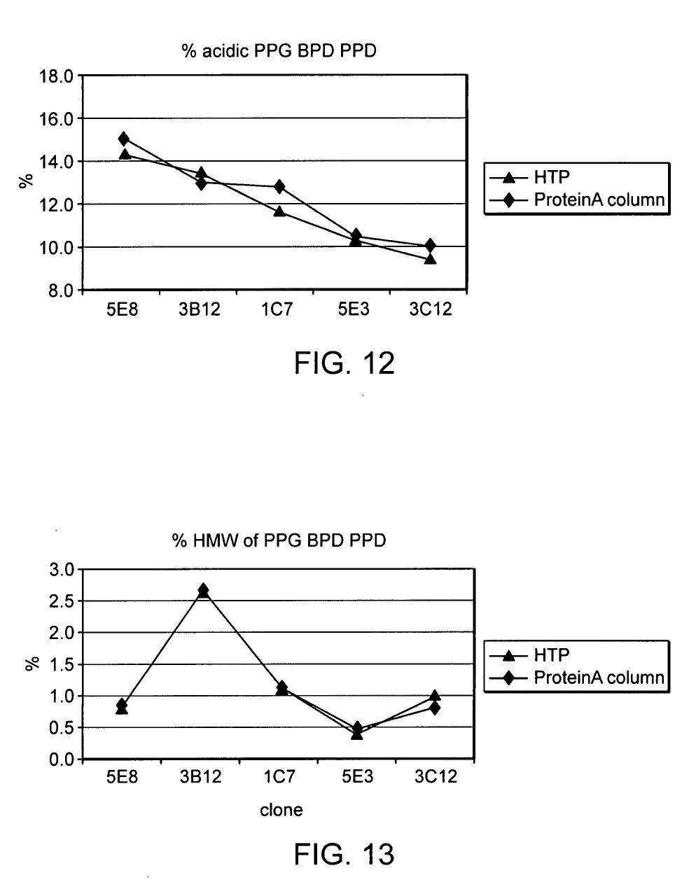

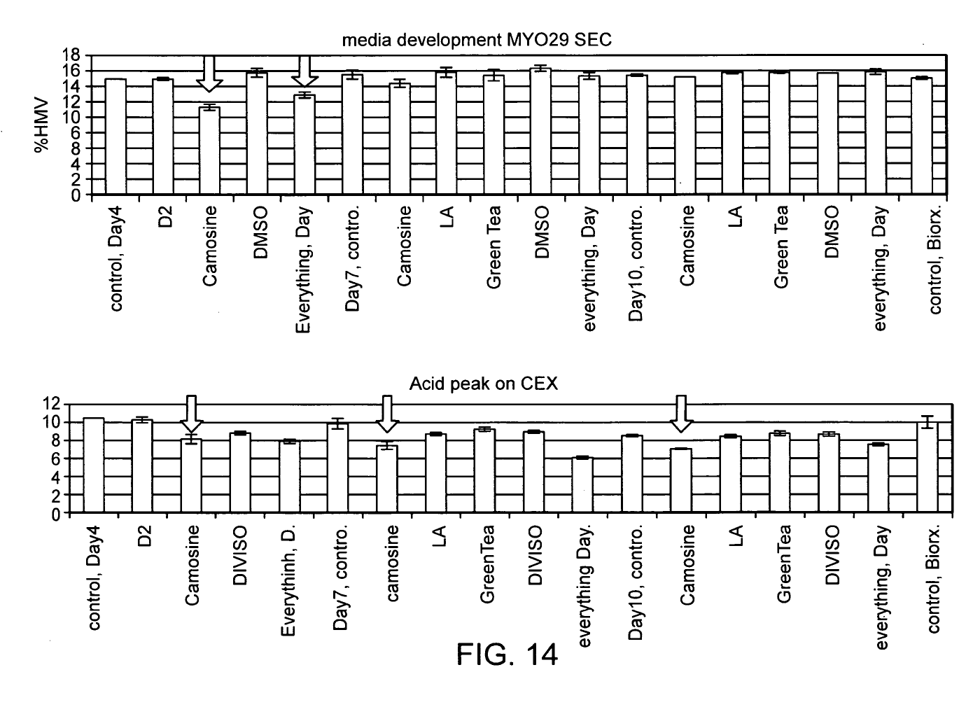

High-throughput purification of samples using the above techniques allowed for rapid purification of up to 96 samples per plate. The purification techniques produced rapid purification of samples without a decrease in the level of purification (FIGS. 12 and 13). As determined by the amount of high molecular weight protein present in each sample, the high-throughput purification techniques performed as well as other techniques (FIGS. 12 and 13). In particular, when comparing the high-throughput purification to standard Protein A purification (FIGS. 12 and 13).

Furthermore, the amount of high molecular weight protein was used to determine the quality of protein generated by cells grown in different cell culture conditions (FIG. 14). As shown in FIG. 14, the various media tested showed different quantities of high molecular weight protein after purification that appeared to be dependent on the media. In particular, certain media conditions showed statistically significant improvements in the amount of high molecular weight protein as compared to the other media (FIG. 14, large arrows). Therefore, the cell culture process development procedure utilized above identified media that showed improved growth characteristics for downstream purification.

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of high-throughput screening of cell lines for protein expression, comprising:
a) screening samples obtained from cell lines to determine a level of expression for a protein-of-interest in each cell line by contacting the samples with a first binding agent;
b) contacting the protein-of-interest with a second binding agent operably linked to a detectable label;
c) determining the level of expression for the protein-of-interest;
d) determining an appropriate quality for the protein-of-interest;
e) selecting a cell line for high-throughput protein expression of the protein-of-interest;
wherein a cell line is selected if the cell line produces a desired level of expression and an appropriate quality for the protein-of-interest.

2. The method of claim 1, wherein the protein-of-interest is selected from the group consisting of antibodies, ligands, receptors, subunits of proteins, fragments of proteins, fusion proteins, recombinant proteins, and fragments of the same.

3. The method of claim 2, wherein the protein-of-interest is an antibody, a recombinant antibody, or a F(ab′)2 fragment.

4. The method of claim 1, wherein the first binding agent is selected from the group consisting of antibodies, ligands, receptors, fusion proteins, subunits of proteins, recombinant proteins, and fragments of the same.

5. The method of claim 4, wherein the first binding agent is Protein A or streptavidin.

6. The method of claim 4, wherein the first binding agent can be attached to a solid support selected from the group consisting of beads, plates, and microarray chips.

7. The method of claim 6, wherein the solid support comprises cellulose, sepharose, polyacrylamide, glass, or polystyrene.

8. The method of claim 1, wherein an appropriate quality for the protein-of-interest is selected from the group consisting of charge, size, enzymatic activity, antibody-epitope interaction, nucleic acid binding, carbohydrate content, secondary structure, tertiary structure, and binding activity.

9. The method of claim 1, wherein the second binding agent is selected from the group consisting of antibodies, ligands, receptors, fusion proteins, subunits of proteins, recombinant proteins, and fragments of the same.

10. The method of claim 9, wherein the second binding agent is an antibody or fragments of the same.

11. The method of claim 10, the antibody is a F(ab′)2 fragment,

12. The method of claim 11, wherein the F(ab′)2 fragment specifically binds to the Fc portion of an antibody.

13. The method of claim 1, wherein the ruthenium labeled second binding agent is labeled with a second detectable label selected from the group consisting of fluorophores, chemical dyes, radioactive binding agents, chemiluminescent binding agents, electrochemiluminescent agents, magnetic binding agents, paramagnetic binding agents, promagnetic binding agents, enzymes that yield a colored product, enzymes that yield a chemiluminescent product, and enzymes that yield a magnetic product.

14. The method of claim 12, wherein the F(ab′)2 fragment is operably linked to two or more ruthenium labels.

15. The method of claim 1, wherein the samples are contacted by a third binding agent attached to a resin.

16. The method of claim 15, wherein the third binding agent is selected from the group consisting of antibodies, ligands, receptors, fusion proteins, subunits of proteins, recombinant proteins, and fragments of the same.

17. The method of claim 16, wherein the third binding agent is Protein A or streptavidin.

18. The method of claim 17, wherein the third binding agent is attached to a solid support.

19. The method of claim 1, wherein the resin is isolated from the mixture and the expressed protein-of-interest is eluted from the third binding agent.

20. The method of claim 19, wherein the protein-of-interest is eluted by a method selected from the group consisting of vacuum elution and gravity flow.

21. The method of claim 1, wherein the cell lines are screened using an automated workstation.

22. A method of high-throughput screening of cell lines for protein expression and production, comprising:
a) contacting a solid support with a sample isolated from a cell line, the solid support having a first binding agent attached to its surface, the first binding agent being capable of binding to a protein-of-interest;
b) contacting the sample with a second binding agent that binds to the protein-of-interest, the second binding agent being operably linked to a detectable label;
c) determining the level of expression of the protein-of-interest by detecting the label operably linked to the second binding agent that is bound to the protein-of-interest; and
d) comparing the level of expression of the protein-of-interest in each cell line to the average level of expression of the protein-of-interest and selecting the cell line based on the comparison,
wherein a cell line is selected for protein production if the level of expression of the protein-of-interest in the cell line is either greater than or less than the average level of expression of the protein-of-interest in all cell lines.

23. The method of claim 22 further comprising isolating supernatants from the selected cell lines, and contacting the supernatants with a reagent that binds to the protein-of-interest.

24. The method of claim 23, wherein the reagent is attached to a solid support.

25. The method of claim 24, wherein the solid support is a multiwell plate.

26. The method of claim 23, wherein the bound protein-of-interest is eluted from the reagent and assayed for appropriate quality.

27. The method of claim 26, wherein the cell line is selected for protein expression if the cell line is selected in step e), and the expressed protein-of-interest has the appropriate quality.

28. The method of claim 22, wherein the protein-of-interest is selected from the group consisting of antibodies, ligands, receptors, subunits of proteins, fragments of proteins, fusion proteins, recombinant proteins, and fragments of the same.

29. The method of claim 28, wherein the protein-of-interest is an antibody, a recombinant antibody, or a F(ab′)2 fragment.

30. The method of claim 22, wherein the first binding agent is selected from the group consisting of antibodies, ligands, receptors, fusion proteins, subunits of proteins, recombinant proteins, and fragments of the same.

31. The method of claim 30, wherein the first binding agent is Protein A or streptavidin.

32. The method of claim 22, wherein the binding agents can be attached to a solid support selected from the group consisting of beads, plates, and microarray chips.

33. The method of claim 24, wherein the solid support comprises cellulose, sepharose, polyacrylamide, glass, or polystyrene.

34. The method of claim 22, wherein the second binding agent is selected from the group consisting of antibodies, ligands, receptors, fusion proteins, subunits of proteins, recombinant proteins, and fragments of the same.

35. The method of claim 34, wherein the second binding agent is an antibody or fragments of the same.

36. The method of claim 35, wherein the antibody is a F(ab′)2 fragment.

37. The method of claim 35, wherein the antibody is an F(ab′)2 fragment that specifically binds to the Fc portion of an antibody.

38. The method of claim 22, wherein the detectable label is selected from the group consisting of fluorophores, chemical dyes, radioactive binding agents, chemiluminescent binding agents, electrochemiluminescent agents, magnetic binding agents, paramagnetic binding agents, promagnetic binding agents, enzymes that yield a colored product, enzymes that yield a chemiluminescent product, and enzymes that yield a magnetic product.

39. The method of claim 38, wherein the detectable label is ruthenium.

40. The method of claim 22, wherein a reagent comprises a resin that has a third binding agent attached to it, the third binding agent being capable of binding to the protein-of-interest.

41. The method of claim 40, wherein the third binding agent is selected from the group consisting of antibodies, ligands, receptors, fusion proteins, subunits of proteins, recombinant proteins, and fragments of the same.

42. The method of claim 41, wherein the third binding agent is Protein A or streptavidin.

43. The method of claim 40, wherein the resin is isolated, and the protein-of-interest is eluted from the third binding agent.

44. The method of claim 43, wherein the protein-of-interest is eluted from the third binding agent using a method selected from the group consisting of vacuum elution and gravity flow.

45. The method of claim 22, wherein the screening of the incubated cell lines utilizes an automated workstation.

46. A method of cell culture process development, comprising:
a) incubating each cell line in a different cell culture condition;
b) contacting each cell line sample with first binding agent attached to a solid support, the first binding agent binding to a protein-of-interest in the cell line samples;
c) contacting the protein-of-interest bound by the first binding agent with a second binding agent, which is operably linked to a detectable label;
d) determining the level of expression for the protein-of-interest by detecting the label operably linked to the second binding agent that is bound to the protein-of-interest; and
e) selecting the cell line based on the detected level of expression of the protein-of-interest,
wherein a cell line is selected if the level of expression of the protein-of-interest in the cell line is greater than or less than the average level of expression of the protein-of-interest in all of the cell lines.

47. The method of claim 46 further comprising isolating supernatants from the selected cell lines, and contacting the supernatants with a reagent that binds to the protein-of-interest.

48. The method of claim 47, wherein the reagent is attached to a solid support.

49. The method of claim 48, wherein the solid support is a multiwell plate.

50. The method of claim 47, wherein the bound protein-of-interest is eluted from the reagent and assayed for appropriate quality.

51. The method of claim 50, wherein the cell line is selected for protein expression if the cell line is selected in step e), and the expressed protein-of-interest has the appropriate quality.

52. The method of claim 46, wherein the protein-of-interest is selected from the group consisting of antibodies, ligands, receptors, subunits of proteins, fragments of proteins, fusion proteins, recombinant proteins, and fragments of the same.

53. The method of claim 52, wherein the protein-of-interest is an antibody, a recombinant antibody, or a F(ab′)2 fragment.

54. The method of claim 46, wherein the first binding agent is selected from the group consisting of antibodies, ligands, receptors, fusion proteins, subunits of proteins, recombinant proteins, and fragments of the same.

55. The method of claim 54, wherein the first binding agent is Protein A or streptavidin.

56. The method of claim 46, wherein the binding agents can be attached to a solid support selected from the group consisting of beads, plates, and microarray chips.

57. The method of claim 48, wherein the solid support comprises cellulose, sepharose, polyacrylamide, glass, or polystyrene.

58. The method of claim 46, wherein the second binding agent is selected from the group consisting of antibodies, ligands, receptors, fusion proteins, subunits of proteins, recombinant proteins, and fragments of the same.

59. The method of claim 58, wherein the second binding agent is an antibody or fragments of the same.

60. The method of claim 59, wherein the antibody is a F(ab′)2 fragment.

61. The method of claim 59, wherein the antibody is an F(ab′)2 fragment that specifically binds to the Fc portion of an antibody.

62. The method of claim 46, wherein the detectable label is selected from the group consisting of fluorophores, chemical dyes, radioactive binding agents, chemiluminescent binding agents, electrochemiluminescent agents, magnetic binding agents, paramagnetic binding agents, promagnetic binding agents, enzymes that yield a colored product, enzymes that yield a chemiluminescent product, and enzymes that yield a magnetic product.

63. The method of claim 62, wherein the detectable label is ruthenium.

64. The method of claim 46, wherein a reagent comprises a resin that has a third binding agent attached to it, the third binding agent being capable of binding to the protein-of-interest.

65. The method of claim 64, wherein the third binding agent is selected from the group consisting of antibodies, ligands, receptors, fusion proteins, subunits of proteins, recombinant proteins, and fragments of the same.

66. The method of claim 65, wherein the third binding agent is Protein A or streptavidin.

67. The method of claim 64, wherein the resin is isolated, and the protein-of-interest is eluted from the third binding agent.

68. The method of claim 67, wherein the protein-of-interest is eluted from the third binding agent using a method selected from the group consisting of vacuum elution and gravity flow.

69. The method of claim 46, wherein the screening of the incubated cell lines utilizes an automated workstation.
